SEQUENCE LISTING (6 sequences)

SEQ ID NO: 1 - DNA, 21 bp, Artificial Sequence
  Synthetic firefly luciferase siRNA sense
cuuacgcugaguacuucgatt

SEQ ID NO: 2 - DNA, 21 bp, Artificial Sequence
  Synthetic firefly luciferase siRNA anti-sense
ucgaaguacucagcguaagtt

SEQ ID NO: 3 - DNA, 29 bp, Artificial Sequence
  Synthetic gp46 siRNA sense
guuccaccauaagaugguagacaacagtt

SEQ ID NO: 4 - DNA, 29 bp, Artificial Sequence
  Synthetic gp46 siRNA anti-sense
guugucuaccaucuuaugguggaacautt

SEQ ID NO: 5 - DNA, 29 bp, Artificial Sequence
  Synthetic scrambled siRNA sense
cgauucgcuagaccggcuucauugcagtt

SEQ ID NO: 6 - DNA, 29 bp, Artificial Sequence
  Synthetic scrambled siRNA anti-sense
gcaaugaagccggucuagcgaaucgautt

(19) United States
(12) Patent Application Publication
(10) Pub. No.: US 2014/0255317 A1
(43) Pub. Date: Sep. 11, 2014

(54) PREPARATION COMPRISING HEXOSE-6-PHOSPHATE-MODIFIED CHOLESTEROL DERIVATIVE

(71) Applicants: Keita Un, Kyoto-shi (JP); Mitsuru Hashida, Kyoto-shi (JP); Shigeru Kawakami, Kyoto-shi (JP); Makoto Kiso, Gifu-shi (JP); Akiharu Ueki, Gifu-shi (JP); Hiromune Ando, Gifu-shi (JP)

(72) Inventors: Keita Un, Kyoto-shi (JP); Mitsuru Hashida, Kyoto-shi (JP); Shigeru Kawakami, Kyoto-shi (JP); Makoto Kiso, Gifu-shi (JP); Akiharu Ueki, Gifu-shi (JP); Hiromune Ando, Gifu-shi (JP)

(73) Assignee: KYOTO UNIVERSITY, Kyoto-shi, Kyoto (JP)

(21) Appl. No.: 14/343,318

(22) PCT Filed: Sep. 5, 2012
(86) PCT No.: PCT/JP2012/072651

§ 371 Date: May 1, 2014

(30) Foreign Application Priority Data
Sep. 7, 2011 (JP) 2011-195229

(51) Int. Cl. A61K 47/28; C12N 15/113; A61K 31/704; A61K 49/00; C07K 14/00; C07J 41/00; A61K 9/127

(52) U.S. Cl. CPC A61K 47/28; C07J 41/0055; C12N 15/113; A61K 9/127; A61K 49/0084; C07K 14/00; A61K 31/704
USPC 424/9.61; 536/5; 514/44 A; 424/450; 514/1.1; 514/4.3; 514/19.3; 514/6.9; 514/34

(57) ABSTRACT

Provided is a compound represented by the general formula (1) (where: G represents a hexose-6-phosphate residue; and L represents a divalent linker group).

### TECHNICAL FIELD

The present invention relates to a hexose-6-phosphate-modified cholesterol derivative-containing formulation.

### BACKGROUND ART

Cancer is a disease known as one of the major causes of death in developed countries. Also in Japan, cancer has been the leading cause of death since around 1980, and the number of deaths due to cancer is predicted to increase in the future. In association with this, development of anticancer agents has been rapidly advanced in the world, and anticancer agents having various action mechanisms have been clinically used. However, an excellent therapeutic effect has not been necessarily found in some cancer treatments. In addition, it is known that as a result of progression of a chronic liver disease such as viral hepatitis or alcoholic liver injury, hepatocytes are replaced by a fibrous tissue through their death and loss, and a liver function attenuates, leading to hepatic cirrhosis. Also in Japan, although there are about 400,000 patients with hepatic cirrhosis, only liver transplantation is performed as radical treatment of hepatic cirrhosis at present. In order that a low-molecular-weight pharmaceutical agent or a nucleic acid pharmaceutical agent may exhibit an excellent therapeutic effect on such intractable disease, it is essential to develop a technology for selectively and efficiently delivering a pharmaceutical agent to target cells depending on diseases. However, it is difficult to deliver a drug or a nucleic acid compound to, for example, target cells present at a low proportion in a tissue, such as hepatic stellate cells in hepatic cirrhosis treatment, or cancer cells in a solid tumor involving difficulty in efficient delivery. Therefore, development of a method of efficiently delivering a low-molecular-weight pharmaceutical agent or a nucleic acid pharmaceutical agent specifically to hepatic stellate cells as main target cells in hepatic cirrhosis treatment or cancer cells is given as an issue.

Patent Literature 1 discloses pharmaceutical compositions for promoting the healing of wounds or fibrotic disorders, in particular for promoting the healing of wounds or fibrotic disorders with reduced scarring.

Patent Literature 2 discloses a liver-directed liposome composition containing a complex that includes a liposome constituted of a sugar-modified cholesterol derivative, and an oligonucleotide.

Non Patent Literature 1 discloses that hepatic cirrhosis is treated by delivering siRNA against gp46 involved in collagen production by means of a liposome targeting a vitamin A receptor expressed in hepatic stellate cells.

Non Patent Literature 2 discloses that cancer is treated by delivering, by means of human serum albumin to which mannose-6-phosphate and doxorubicin as an anticancer agent are bound, doxorubicin to cancer cells expressing a mannose-6-phosphate receptor.

Non Patent Literature 3 discloses that hepatic cirrhosis is treated by delivering, by means of human serum albumin to which mannose-6-phosphate and doxorubicin as an anticancer agent are bound, doxorubicin to hepatic stellate cells expressing a mannose-6-phosphate receptor.

Patent Literature 1 involves a problem in that a mannose-6-phosphate analog is a low-molecular-weight compound, and hence after intravenous administration to a living body, diffuses into all of the tissues in the body, resulting in low distribution to liver as a target organ and low efficiency of delivery to hepatic stellate cells as target cells.

Patent Literature 2 involves a problem in terms of a characteristic of selective distribution and efficiency of delivery to hepatic stellate cells or the like.

Non Patent Literature 1 involves problems, for example, in that: the vitamin A receptor is also expressed on surfaces of normal hepatic stellate cells, and hence toxicity is exhibited on hepatic stellate cells having normal functions; and when a vitamin A-modified liposome is administered in a large amount or administered at frequent intervals, hypervitaminosis A may develop.

Non Patent Literatures 2 and 3 involve problems, for example, in that: the number of doxorubicin molecules that can bind to one molecule of albumin is limited, and hence an amount of doxorubicin to be delivered to target cells is low with respect to a dose of the formulation and it is necessary to administer the formulation at an extremely high dose in order to express a therapeutic effect; and the kind of a drug that can bind to albumin is limited, and hence the application range is narrow.

### CITATION LIST

### Patent Literature

[PTL 1] JP 11-510179 A
[PTL 2] JP 2007-112768 A

### Non Patent Literature

[NPL 1] Niitsu et. al., Nat. Biotechnol. Vol. 26, pp 431-442, 2008.
[NPL 2] Prakash et. al., Int. J. Cancer, Vol. 126, pp 1966-1981, 2010.
[NPL 3] Greupink et. al., J. Pharmacol. Exp. Ther., Vol. 317, pp 514-521, 2006.

### SUMMARY OF INVENTION

### Technical Problem

An object of the present invention is to efficiently deliver a low-molecular-weight compound, a protein, or a nucleic acid compound into mannose-6-phosphate receptor-expressing cells such as hepatic stellate cells under the condition of hepatic cirrhosis or cancer cells.

### Solution to Problem

According to one embodiment of the present invention, the following mannose-6-phosphate-modified cholesterol derivative-containing formulation is provided.

Item 1. A compound, which is represented by the following general formula (1):

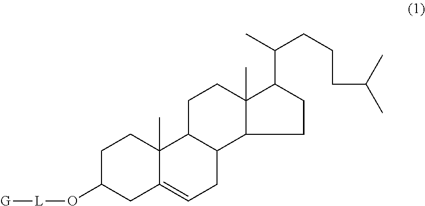

where: G represents a hexose-6-phosphate residue; and L represents a divalent linker group.

Item 2. A compound according to Item 1, in which G represents a mannose-6-phosphate residue, a galactose-6-phosphate residue, a glucose-6-phosphate residue, or a fructose-6-phosphate residue.
Item 3. A compound according to Item 1 or 2, in which the linker group is represented by the following general formula:

—X—(CH₂)m-NHCO(CH₂)n-NHCO—

where: X represents S or O; m represents an integer of from 2 to 6; and n represents an integer of from 2 to 6.
Item 4. A hexose-6-phosphate-modified cholesterol derivative-containing formulation, including: a liposome including the compound according to anyone of Items 1 to 3; and a physiologically active substance complexed with the liposome.
Item 5. A formulation according to Item 4, in which the physiologically active substance includes a therapeutic drug for hepatic cirrhosis, hepatitis, hepatic fibrosis, cancer, diabetes, a lysosomal disease, or the like.

Item 6. A formulation according to Item 4 or 5, in which the physiologically active substance is a drug, a protein, or a nucleic acid.

Item 7. A formulation according to any one of Items 4 to 6, in which the physiologically active substance is an anticancer agent, plasmid DNA/RNA, antisense DNA, an aptamer, siRNA, shRNA, or miRNA.

Item 8. A formulation according to any one of Items 4 to 6, in which the physiologically active substance includes an organic fluorescent dye.

### Advantageous Effects of Invention

According to the formulation of the present invention, the low-molecular-weight compound, the protein, or the nucleic acid compound can be efficiently delivered to hexose-6-phosphate receptor-expressing cells such as mannose-6-phosphate receptor-expressing cells distributed in a living body. According to one embodiment of the present invention, the efficient delivery of the drug, the protein, or the nucleic acid compound into hexose-6-phosphate receptor-expressing cells, in particular, mannose-6-phosphate receptor-expressing cells such as hepatic stellate cells under the condition of hepatic cirrhosis or cancer cells can be achieved by forming a complex with the low-molecular-weight compound, the protein, or the nucleic acid compound in the derivative-containing formulation and then administering the formulation to a living body. Herein, examples of the drug include a pharmaceutical agent, a fluorescent substance, and a peptide. Examples of the protein include an enzyme, a hormone, and a cytokine. Examples of the nucleic acid compound include DNA and RNA. Examples of the DNA include plasmid DNA and antisense DNA. Examples of the RNA include siRNA, shRNA, miRNA, and antisense RNA. The base sequence of the nucleic acid compound is not particularly limited. According to one embodiment of the present invention, the low-molecular-weight compound, the protein, or the nucleic acid compound can be delivered under a non-invasive condition to, for example, hexose-6-phosphate receptor-expressing cells such as cells present at a low proportion in a tissue, e.g., mannose-6-phosphate receptor-expressing cells such as hepatic stellate cells or cancer cells involving difficulty in selective delivery. Accordingly, the loading of a known or novel pharmaceutical agent into the formulation of the present invention enables the development as a high-functionality formulation for hepatic cirrhosis, hepatitis, hepatic fibrosis, cancer, diabetes, or a lysosomal disease, which is difficult to completely cure. Therefore, the present invention has high applicability as a technology for delivering a pharmaceutical agent in drug and gene treatments.

In Japan and overseas countries, there are many patients with hepatic cirrhosis, hepatitis, hepatic fibrosis, cancer, diabetes, and a lysosomal disease. However, there is no efficient method of delivering a drug or a nucleic acid to target cells depending on diseases, and the research and development of pharmaceutical agents that allow the complete cure of the diseases have not been advanced. There is a report that a hexose-6-phosphate receptor such as a mannose-6-phosphate receptor is expressed on surfaces of the target cells. However, a phosphoric acid ester bond is poor in chemical stability and a compound of interest containing cholesterol as a hydrophobic moiety and a phosphate group as a hydrophilic moiety is amphiphilic. Accordingly, in the related art, it has been difficult to synthesize a hexose-6-phosphate-modified derivative that can be formulated. In the present invention, the inventors of the present invention have solved the above-mentioned problems, succeeded in synthesizing a hexose-6-phosphate-modified cholesterol derivative, and enabled applications of a formulation containing the derivative to treatments for the diseases.

### BRIEF DESCRIPTION OF DRAWINGS

FIG. 1 illustrates a synthetic pathway for a mannose-6-phosphate-modified cholesterol derivative.

FIG. 2 shows the evaluation of mannose-6-phosphate-modified cholesterol derivative-containing liposomes for physical properties.

FIG. 3 shows the evaluation of mannose-6-phosphate-modified cholesterol derivative-containing emulsions for physical properties.

FIG. 4 show the evaluation of mannose-6-phosphate-modified cholesterol derivative-containing liposomes for intracellular uptake characteristics.

FIG. 5 show the characteristics of distribution of mannose-6-phosphate-modified cholesterol derivative-containing liposomes into a tumor (left figure) and liver (right figure) under the condition of hepatic cirrhosis.

FIG. 6 shows the evaluation of a mannose-6-phosphate-modified cholesterol derivative-containing liposome/siRNA complex for physical properties.

FIG. 7 shows the tumor distribution characteristics of siRNA by mannose-6-phosphate-modified cholesterol derivative-containing liposome/siRNA complexes.

FIG. 8 show the suppressing effects of mannose-6-phosphate-modified cholesterol derivative-containing liposome/siRNA complexes on gene expression in tumor tissues.

FIG. 9 show the suppressing effects of mannose-6-phosphate-modified cholesterol derivative-containing liposome/gp46 siRNA complexes on gp46 expression in liver.

FIG. 10 show the suppressing effects of mannose-6-phosphate-modified cholesterol derivative-containing liposome/gp46 siRNA complexes on various hepatic cirrhosis markers.

FIG. 11 shows the hepatic distribution characteristics of doxorubicin by a doxorubicin-encapsulated mannose-6-phosphate-modified cholesterol derivative-containing liposome.

FIG. 12 show the suppressing effects of a doxorubicin-encapsulated mannose-6-phosphate-modified cholesterol derivative-containing liposome on hepatic cirrhosis markers.

FIG. 13 shows the tumor tissue distribution characteristics of doxorubicin by a doxorubicin-encapsulated mannose-6-phosphate-modified cholesterol derivative-containing liposome.

FIG. 14 shows the antitumor effect of a doxorubicin-encapsulated mannose-6-phosphate-modified cholesterol derivative-containing liposome.

FIG. 15 shows the preparation of an indocyanine green (ICG)-encapsulated M6P-modified liposome. M6P=0% before and after filtration and M6P=15% before and after filtration are shown in this order from the left of the figure.

FIG. 16 shows the preparation of a hematoporphyrin (Hp)-encapsulated M6P-modified liposome. M6P=15% after and before filtration and M6P=0% after and before filtration are shown in this order from the left of the figure.

### DESCRIPTION OF EMBODIMENTS

In one embodiment of the present invention, there is provided a compound of the following general formula (1):

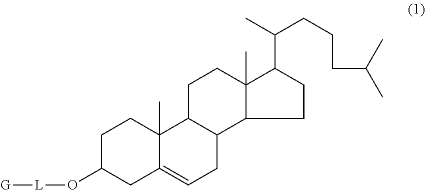

(In the formula: G represents a hexose-6-phosphate residue; and L represents a divalent linker group). The compound of the general formula (1) has a structure in which a hexose-6-phosphate residue is bonded to a hydroxy group at the 3-position of cholesterol via a linker group.

The hexose is, for example, a hexose having a primary hydroxy group (—CH₂OH group) at the 6-position, such as mannose, galactose, glucose, or fructose, and the hexose-6-phosphate residue is a residue in which the hydroxy group at the 6-position has been converted to a phosphoric acid ester.

The divalent linker group is a divalent group present between the 1-position of the hexose-6-phosphate and the hydroxy group at the 3-position of cholesterol, and is, for example, a group that is bonded to the 1-position of the hexose-6-phosphate via a sulfur atom (S) or an oxygen atom (O) and is bonded to the hydroxy group at the 3-position of cholesterol via an ether bond (—O—), an ester bond (—O—CO—), or a urethane bond (O—CO—NH).

An example of the divalent linker group is a group represented by —X—R—Y—. In the formula, X represents O or S.

Y represents, for example: an alkylene group having from 1 to 6 carbon atoms such as —(CH₂)—, —(CH₂CH₂)—, —(CH₂CH₂CH₂)—, or —(CH₂CH₂CH₂CH₂)—; a cycloalkylene group having from 3 to 6 carbon atoms (such as a 1,3-cyclopentylene group or a 1,4-cyclohexylene group); an arylene group (such as 1,3-phenylene or 1,4-phenylene); an aralkylene group (such as 1,3-xylylene, 1,4-xylylene, 1,3-benzylidene, or 1,4-benzylidene); —NHCO—; —O—CO—; or —CO—.

When Y represents an alkylene group having from 1 to 6 carbon atoms, a cycloalkylene group having from 3 to 6 carbon atoms, an arylene group, or an aralkylene group, R represents a single bond or R1-R2 (where R1 represents an alkylene group having from 1 to 6 carbon atoms, a cycloalkylene group having from 3 to 6 carbon atoms, an arylene group, or an aralkylene group and R2 represents —NHCO—, —CONH—, —O—, —S—, —NHCOO—, —OCONH—, —CO—, —COO—, or —O—CO—) or a polyether group (such as —(CH₂CH₂O)ₙ₁— (n1 represents an integer of from 1 to 20)). When Y represents —NHCO—, —O—CO—, or —CO—, R represents R1 or R1-R2-R1 (where R1's are identical to or different from each other and each represent an alkylene group having from 1 to 6 carbon atoms, a cycloalkylene group having from 3 to 6 carbon atoms, an arylene group, or an aralkylene group and R2 represents —NHCO—, —CONH—, —O—, —S—, —NHCOO—, —OCONH—, —CO—, —COO—, or —O—CO—).

The divalent linker group is preferably a group represented by the following general formula:

—X—(CH₂)m-NHCO(CH₂)n-NHCO—

(where X represents S or O, m represents an integer of from 2 to 6, preferably 2 or 3, and n represents an integer of from 2 to 6, preferably 2 or 3).

A specific example of the divalent linker group is —S—(CH₂)m-NHCO(CH₂)n-NHCO— (m and n each represent an integer of from 1 to 6), —S—(CH₂CH₂O)ₙ₁—CH2CH2-, or —O—(CH₂CH₂O)ₙ₁—CH2CH2- (n1 represent an integer of from 1 to 20).

The particle diameter of a liposome is from about 30 to 200 nm, preferably from about 50 to 150 nm, particularly preferably from about 70 to 120 nm. The liposome to be used in the present invention may be any of a multi-layered liposome and a single-layered liposome. The liposome is produced by a sonication method, a reverse phase evaporation method, a freeze-thawing method, a lipid dissolution method, a spray drying method, or the like, and contains a phospholipid, a glycolipid, a sterol, a glycol, a cationic lipid, a lipid having a polyethylene glycol group (e.g., a PEG-phospholipid), or the like.

Herein, the term “complexation” means that the liposome and a physiologically active substance are integrated (move integrally). The term is meant to encompass, for example, a case where the physiologically active substance is encapsulated into the liposome, a case where the physiologically active substance is adsorbed or bound to a lipid membrane surface (inner surface or outer surface) of the liposome, a case where part of the physiologically active substance enters a lipid membrane, and a case where the physiologically active substance permeates a lipid membrane. The physiologically active substance is adsorbed or bound to the lipid membrane via an ionic bond, a hydrogen bond, a hydrophobic interaction, or the like. For example, the ionic bond is a bond via an ionic bond between a cation or anion of a constituent of the liposome and an anion or cation of the physiologically active substance.

A neutral phospholipid contained in the liposome of the present invention may be preferably exemplified by lecithins obtained from soybeans, egg yolk, and the like, lysolecithins, and/or derivatives of hydrogenated products and hydroxides thereof.

Other phospholipids are exemplified by phosphatidylcholine (PC), phosphatidylserine (PS), phosphatidylethanolamine (PE), cardiolipin, sphingosine, ceramide, sphingomyelin, ganglioside, sphingophospholipid, egg yolk lecithin, hydrogenated egg yolk lecithin, soybean lecithin, or hydrogenated soybean lecithin, which is constituted of a saturated or unsaturated fatty acid having n carbon atoms (n represents an integer of from 3 to 30) derived from egg yolk, soybeans, or other animals and plants or synthesized.

A charged lipid may be incorporated as a constituent of the lipid membrane constituting the liposome of the present invention, and the liposome may be manufactured by using, as an anionic lipid, phosphatidylinositol, phosphatidylglycerol, or the like, which is constituted of a saturated or unsaturated fatty acid having n2 carbon atoms (n2 represents an integer of from 3 to 30).

As an anionic lipid membrane component constituting the lipid membrane of the liposome, in addition to a negatively charged phospholipid such as phosphatidylinositol or phosphatidylglycerol, there may be given, for example, phosphatidic acid, dicetyl phosphate (DCP), dilauryl phosphate, dimyristyl phosphate, or phosphatidyl glycerol phosphate, which is constituted of a saturated or unsaturated fatty acid having n2 carbon atoms (n2 represents an integer of from 3 to 30).

Examples of the cationic lipid include 3β-[N—(N′,N′-dimethylaminoethane)-carbamoyl]cholesterol (DC-chol), 1,2-dioleoyloxy-3-(trimethylammonium)propane (DOTAP), N,N-dioctadecylamidoglycylspermine (DOGS), dimethyldioctadecylammonium bromide (DDAB), N-[1-(2,3-dioleyloxy)propyl]-N,N,N-trimethylammonium chloride (DOTMA), 2,3-dioleyloxy-N-[2(spermine-carboxamido)ethyl]-N,N-dimethyl-1-propanaminium trifluoroacetate (DOSPA), and N-[1-(2,3-dimyristyloxy)propyl]-N,N-dimethyl-N-(2-hydroxyethyl)ammonium bromide (DMRIE), as well as an ester between dipalmitoylphosphatidic acid (DPPA) and hydroxyethylenediamine, and an ester between distearoylphosphatidic acid (DSPA) and hydroxyethylenediamine.

Examples of the glycolipid may include: glycerolipids such as digalactosyl diglyceride and galactosyldiglyceridesulfate; and sphingoglycolipids such as galactosylceramide, galactosylceramide sulfate, lactosylceramide, ganglioside G7, ganglioside G6, and ganglioside G4.

The anionic lipid or the cationic lipid has only to be added so as to be contained at a ratio of from 0.1 to 15 mass % with respect to the total lipid amount, preferably from 1 to 10 mass % with respect to the total lipid amount, more preferably from 5 to 10 mass % with respect to the total lipid amount.

As a constituent of the liposome membrane, any other substance may be added in addition to the lipid as required. Examples thereof include a sterol that acts as a lipid membrane stabilizer such as cholesterol, sitosterol, campesterol, brassicasterol, ergosterol, desmosterol, zymosterol, stigmasterol, lathosterol, lanosterol, dehydroepiandrosterone (DHEA), dihydrocholesterol, cholesterol ester, phytosterol, cholestanol, or a vitamin D; and a hormone.

The ratio of the compound of the general formula (1) in the liposome is from about 1 to 60 wt %, preferably from about 5 to 55 wt %, more preferably from about 10 to 50 wt %, particularly preferably from about 15 to 45 wt %.

Examples of the physiologically active substance to be complexed with the liposome include a nucleic acid, a protein, and a drug. The nucleic acid may be any of DNA and RNA. As the DNA, there is given one expressing a gene, and examples thereof include a plasmid, a gene construct including a gene linked to a promoter, and an artificial gene. Examples of the DNA include gene-expressing plasmid DNA, antisense DNA, an aptamer, and DNA expressing RNA such as siRNA or shRNA. Examples of the RNA include siRNA, antisense RNA, an aptamer, and shRNA.

Examples of the physiologically active substance such as the nucleic acid, the protein, or the drug include: one having a cell-damaging or cell death-inducing action such as cytotoxicity or an apoptosis-inducing action when taken up into cells or expressed in cells; and one having, for example, an inhibiting action on the fibrosis of hepatic stellate cells.

Examples of the drug include an anticancer agent, an anti-allergy agent, an antibacterial agent, an antimycotic agent, an antiviral agent, an immunosuppressive agent, a vaccine, an interferon, an interleukin, a growth factor, a peptide hormone, an enzyme, a steroid hormone, an anti-rheumatic drug, an antigen, an antibody, a receptor, and ligands thereof.

Further, the drug includes a fluorescent substance such as an organic fluorescent dye. Examples of the organic fluorescent dye include indocyanine green, coumarin, rhodamine, xanthene, hematoporphyrin, and fluorescamine. The organic fluorescent dye can be applied to fluorescence imaging of cancer cells.

In addition, the drug may be a drug for a sonodynamic therapy that induces cancer cell death through generation of active oxygen by ultrasonic irradiation. Examples of such drug include indocyanine green, hematoporphyrin, diacetylhematoporphyrin, photofrin II, mesoporphyrin, copper protoporphyrin, tetraphenylporphyrin, ATX-70, ATX-S10, pheophorbide-α, and phthalocyanine.

An example of a manufacturing method for the liposome is specifically described. For example, the phospholipid, cholesterol, and the like describe above are dissolved in an appropriate organic solvent, the solution is charged into an appropriate container, the solvent is distilled off under reduced pressure to form a phospholipid membrane on the inner surface of the container, an aqueous solution, preferably buffer containing a complex is added thereto, and the mixture is stirred. Thus, a liposome encapsulating the complex can be obtained. The liposome is mixed with a nanoparticle of the present invention, which has been subjected to freeze-drying treatment, directly or after having been freeze-dried once. Thus, a composite particle of the liposome and the nanoparticle can be obtained.

The liposome of the present invention has a zeta potential of from about −30 to 50 mV, preferably from about −20 to 30 mV, more preferably from about −15 to 25 mV.

### EXAMPLES

Examples are shown below. However, the present invention is by no means limited to Examples shown below.

### Example 1

### Evaluation for Basic Physical Properties

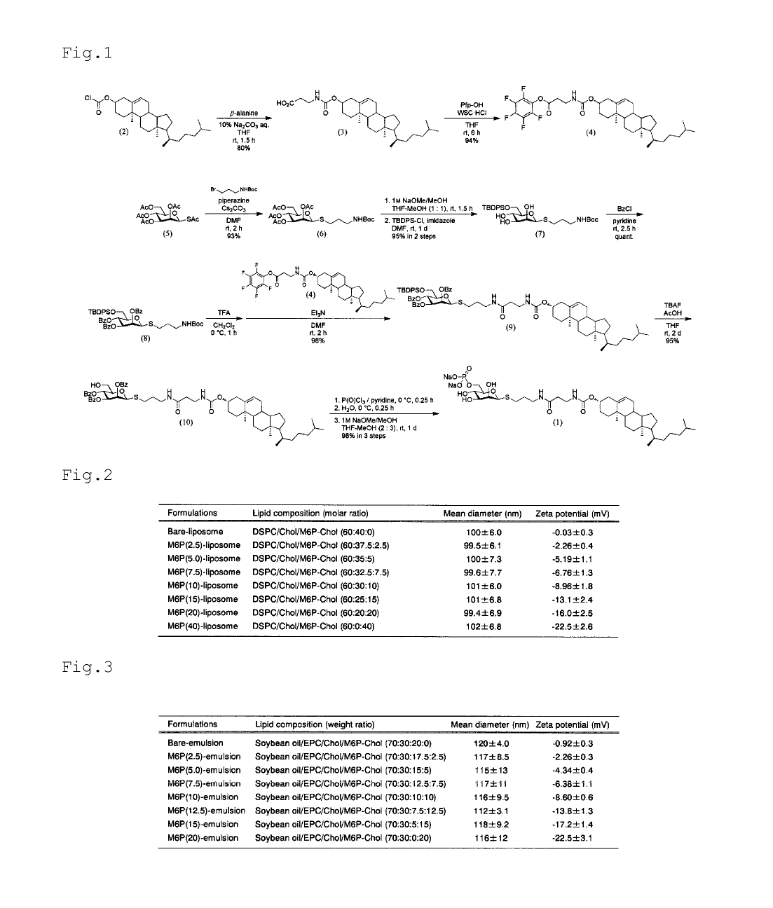

### 1. Synthetic Pathway for Mannose-6-Phosphate-Modified Cholesterol Derivative (FIG. 1)

A mannose-6-phosphate-modified cholesterol derivative is synthesized by a manufacturing method including the following steps. A phosphate group is introduced at the final stage of the synthesis. First, an intermediate (8) in which only the 6-position of mannose as a phosphate-introducing position was protected with a different protection group was synthesized, followed by condensation with a cholesterol derivative (4) synthesized separately, the phosphorylation of the 6-position of mannose, and deprotection. Thus, a final product of interest (1) was synthesized. (In the formulae, THF represents tetrahydrofuran, Pfp represents a pentafluorophenyl group, WSC represents a 1-ethyl-3-(3-dimethylaminopropyl)carbodiimide, Ac represents an acetyl group, Boc represents a tert-butyloxycarbonyl group, DMF represents N,N-dimethylformamide, Me represents a methyl group, TBDPS represents a tert-butyldiphenylsilyl group, Bz represents a benzoyl group, TFA represents trifluoroacetic acid, Et represents an ethyl group, and TBAF represents tetra-n-butylammoniumfluoride.)

### Step [1]: Synthesis of N-cholesteryloxycarbonyl-3-aminopropionic acid (3)

To a solution of cholesteryl chloroformate (2) (2.09 g) in tetrahydrofuran (25 mL) were added R-alanine (0.50 g) and a 10% sodium carbonate aqueous solution (50 mL) at room temperature, and the mixture was stirred at room temperature for 1.5 hours. After neutralization with 2 M hydrochloric acid, the reaction liquid was transferred to a separating funnel and extracted with chloroform. The organic layer was washed with water and brine. The resultant was dried over anhydrous sodium sulfate and then concentrated under reduced pressure. The resultant crude product was purified by silica gel column chromatography. A mixture obtained by elution with a mixed solvent of chloroform and methanol (95:5) and subsequent elution with a mixed solvent of chloroform and methanol (90:10) was purified again by silica gel column chromatography. Elution with chloroform and subsequent elution with a mixed solvent of chloroform and methanol (80:20) gave a compound (3) (1.87 g, 80% yield).

Melting point: 173.5-174.5° C.

[α]_{D}−24.0° (c 0.5, chloroform)

¹H-NMR (500 MHz, CDCl₃): δ 5.37 (1H, m, H-6^{Chol}), 5.14 (1H, br s, NH), 4.49 (1H, m, H-3^{Chol}), 3.44 (2H, q, J=6.0 Hz, NHCH₂), 2.61 (2H, m, COCH₂), 2.34-2.27 (2H, m, H-4^{Chol}), 2.02-1.79 (5H, m, H-1eq^{Chol}, H-2eq^{Chol}, H-7eq^{Chol}, H-12eq^{Chol}, H-16eq^{Chol}), 1.60-0.90 (27H, m, CH^{Chol}, CH₂^{Chol}, CH₃^{Chol}), 0.87 (3H, d, J=6.7 Hz, CH₃CH₂CH₃), 0.86 (3H, d, J=6.6 Hz, CH₃CH₂CH₃), 0.68 (3H, s, H-18^{Chol}).

ESI-TOF (high resolution): calcd for C₃₁H₅₁NNaO₄ [M+Na]⁺: 524.3710. found; 524.3707.

### Step [2]: Synthesis of N-cholesteryloxycarbonyl-3-aminopropionic acid pentafluorophenyl ester (4)

To a solution of the compound (3) (218.0 mg) in dichloromethane (4 mL) were added pentafluorophenol (98.6 mg) and 1-ethyl-3-(3-dimethylaminopropyl)carbodiimide hydrochloride (99.9 mg) under an argon atmosphere at room temperature, and the mixture was stirred at room temperature for 6 hours. The reaction liquid was diluted with ethyl acetate and then transferred to a separating funnel. The organic layer was washed with water and brine. The resultant was dried over anhydrous sodium sulfate and then concentrated under reduced pressure. The resultant crude product was purified by silica gel column chromatography. Elution with a mixed solvent of n-hexane and ethyl acetate (90:10) gave a compound (4) (273.6 mg, 94% yield).

[α]_{D}−14.7° (c 1.1, chloroform)

¹H-NMR (CDCl₃): δ 5.38 (1H, m, H-6^{Chol}), 5.04 (1H, br s, NH), 4.51 (1H, m, H-3^{Chol}), 3.57 (2H, q, J=6.0 Hz, NHCH₂), 2.94 (2H, t, J=6.0 Hz, COCH₂), 2.37-2.26 (2H, m, H-4^{Chol}), 2.03-1.94 (2H, m, H-7eq^{Chol}, H-12eq^{Chol}), 1.89-1.79 (3H, m, H-1eq^{Chol}, H-2eq^{Chol}, H-16eq^{Chol}), 1.60-0.91 (27H, m, CH^{Chol}, CH₂^{Chol}, CH₃^{Chol}), 0.87 (3H, d, J=6.6 Hz, CH₃CH₂CH₃), 0.86 (3H, d, J=6.6 Hz, CH₃CH₂CH₃), 0.68 (3H, s, H-18^{Chol}).

ESI-TOF (high resolution): calcd for C₃₇H₅₀F₅NNaO₄ [M+Na]⁺: 690.3552. found; 690.3551.

### Step [3]: Synthesis of N-(tert-butyloxycarbonyl)-3-aminopropyl 2,3,4,6-tetra-O-acetyl-1-thio-β-D-mannopyranoside (6)

To a solution of 1,2,3,4,6-penta-O-acetyl-1-thio-β-d-mannopyranoside (5) (see Journal of Chemical Society, Perkin Transactions 1, pp. 832-837, 2001) (5.10 g) and N-(tert-butyloxycarbonyl) 3-bromopropylamine (4.48 g) in N,N-dimethylformamide (125 mL) were added cesium carbonate (8.18 g) and piperazine (1.30 g) under an argon atmosphere at room temperature. The mixture was stirred at room temperature for 2 hours. After that, water was added to the reaction liquid, and the mixture was transferred to a separating funnel and extracted with ethyl acetate. The organic layer was washed with 2 M hydrochloric acid, water, and brine. The resultant was dried over anhydrous sodium sulfate and then concentrated under reduced pressure. The resultant crude product was recrystallized from ethyl acetate. Then, the mother liquor was recrystallized again from a mixed solvent of n-hexane and ethyl acetate to give a compound (6) (6.06 g, 93% yield).

Melting point 178.0-179.0° C.

[α]_{D}−50.0° (c 1.0, chloroform)

¹H-NMR (CDCl₃): δ 5.51 (1H, dd, J_{1,2}=0.9 Hz, J_{2,3}=3.5 Hz, H-2), 5.25 (1H, t, J_{3,4}=J_{4,5}=10.1 Hz, H-4), 5.06 (1H, dd, J_{2,3}=3.5 Hz, J_{3,4}=10.1 Hz, H-3), 4.77 (1H, br s, H-1), 4.65 (1H, br s, NH), 4.26 (1H, dd, J_{5,6}=6.0 Hz, J_{gem}=12.3 Hz, H-6), 4.16 (1H, dd, J_{5,6}=2.4 Hz, J_{gem}=12.3 Hz, H-6), 3.70 (1H, ddd, J_{4,5}=10.1 Hz, J_{5,6}=2.4 Hz, J_{5,6}=6.0 Hz, H-5), 3.22 (2H, m, NHCH₂), 2.74 (2H, t, J=7.1 Hz, SCH₂), 2.19 (3H, s, Ac), 2.09 (3H, s, Ac), 2.05 (3H, s, Ac), 1.98 (3H, s, Ac), 1.82 (2H, m, CH₂CH₂CH₂), 1.57 (9H, s, ^{t}Bu).

ESI-TOF (high resolution): calcd for C₂₂H₃₅NNaO₁₁S [M+Na]⁺: 544.1823. found; 544.1824.

### Step [4]: Synthesis of N-(tert-butyloxycarbonyl)-3-aminopropyl 6-O-tert-butyldiphenylsilyl-1-thio-β-D-mannopyranoside (7)

To a solution of the compound (6) (1.50 g) in a mixed solvent of tetrahydrofuran (30 mL) and methanol (30 mL) was added a solution of M sodium methylate in methanol (0.29 mL) under an argon atmosphere at room temperature. The mixture was stirred at room temperature for 1.5 hours. After that, the reaction liquid was neutralized with Dowex-50 (H⁺), filtered, and then concentrated under reduced pressure. To a solution of the resultant crude product in N,N-dimethylformamide (125 mL) were added tert-butyldiphenylchlorosilane (0.90 mL) and imidazole (0.47 g) under an argon atmosphere at room temperature, and the mixture was stirred at room temperature for 1 day. After that, tert-butyldiphenylchlorosilane (0.15 mL) was added thereto, and the mixture was stirred at room temperature for 1 hour. The reaction liquid was diluted with toluene, concentrated under reduced pressure, and then diluted with chloroform and water. The dilution was transferred to a separating funnel, and the aqueous layer was extracted with chloroform. The extracted organic layer was washed with brine. The resultant was dried over anhydrous sodium sulfate and then concentrated under reduced pressure. The resultant crude product was purified by silica gel column chromatography. Elution with a mixed solvent of chloroform and methanol (97:3) gave a compound (7) (1.62 g, 95% yield).

[α]_{D}−2.7° (c 1.0, chloroform)

¹H-NMR (CDCl₃): δ 7.69-7.67 (4H, m, Ar), 7.47-7.38 (6H, m, Ar), 4.65 (1H, br s, H-1), 4.57 (1H, br s, NH), 4.01 (1H, br d, J_{2,3}=3.4 Hz, H-2), 3.93 (2H, d, J_{5,6}=5.3 Hz, H-6), 3.82 (1H, dd, J_{3,4}=9.2 Hz, J_{4,5}=9.4 Hz, H-4), 3.58 (1H, dd, J_{2,3}=3.4 Hz, J_{3,4}=9.2 Hz, H-3), 3.70 (1H, dt, J_{4,5}=9.4 Hz, J_{5,6}=5.3 Hz, H-5), 3.21-3.14 (3H, m, NHCH₂, OH), 2.75-2.65 (3H, m, SCH₂, OH), 1.78 (2H, m, CH₂CH₂CH₂), 1.41 (9H, s, O^{t}Bu), 1.06 (9H, s, Si^{t}Bu).

ESI-TOF (high resolution): calcd for C₃₀H₄₅NNaO₇SSi [M+Na]⁺: 614.2578. found; 614.2577.

### Step [5]: Synthesis of N-(tert-butyloxycarbonyl)-3-aminopropyl 2,3,4-tri-O-benzoyl-6-O-tert-butyldiphenylsilyl-1-thio-β-D-mannopyranoside (8)

To a solution of the compound (7) (1.62 g) in pyridine (25 mL) was added benzoyl chloride (1.90 mL) under an argon atmosphere at 0° C. The mixture was stirred at room temperature for 2.5 hours. After that, excess water was added to quench the reaction, and the mixture was concentrated under reduced pressure. The residue was diluted with ethyl acetate, transferred to a separating funnel, and washed with water and brine. The resultant was dried over anhydrous sodium sulfate and then concentrated under reduced pressure. The resultant crude product was purified by silica gel column chromatography. Elution with a mixed solvent of toluene and ethyl acetate (83:7) and subsequent elution with a mixed solvent at toluene and ethyl acetate (90:10) gave a compound (8) (2.47 g, quant. yield).

[α]_{D}−12.5° (c 1.0, chloroform)

¹H-NMR (CDCl₃): δ 8.11-8.09 (2H, m, Ar), 7.88-7.86 (2H, m, Ar), 7.81-7.77 (4H, m, Ar), 7.60-7.51 (4H, m, Ar), 7.47-7.14 (13H, m, Ar), 6.08 (1H, dd, J_{3,4}=10.2 Hz, J_{4,5}=10.0 Hz, H-4), 5.99 (1H, br d, J_{2,3}=3.4 Hz, H-2), 5.56 (1H, dd, J_{2,3}=3.4 Hz, J_{3,4}=10.2 Hz, H-3), 5.04 (1H, br s, H-1), 4.56 (1H, br s, NH), 3.94-3.83 (3H, m, H-5, H-6), 3.19 (2H, m, NHCH₂), 2.79 (2H, m, SCH₂), 1.85 (2H, m, CH₂CH₂CH₂), 1.42 (9H, s, O^{t}Bu), 1.08 (9H, s, Si^{t}Bu).

ESI-TOF (high resolution): calcd for C₅₁H₅₇NNaO₁₀SSi [M+Na]⁺: 926.3365. found; 926.3364.

### Step [6]: Synthesis of N-(N-cholesteryloxycarbonyl-3-aminopropionyl)-3-aminopropyl 2,3,4-tri-O-benzoyl-6-O-tert-butyldiphenylsilyl-1-thio-β-D-mannopyranoside (9)

To a solution of the compound (8) (162.4 mg) in dichloromethane (3 mL) was slowly added trifluoroacetic acid (1 mL) under an argon atmosphere at 0° C. The mixture was stirred at 0° C. for 1 hour. After that, the reaction liquid was concentrated under reduced pressure. The residue was dissolved with N,N-dimethylformamide (2 mL), and the compound (4) (144.2 mg) was added thereto under an argon atmosphere at 0° C. To the mixed liquid was slowly added triethylamine (0.05 mL), and the mixture was stirred at room temperature for 2 hours. After dilution with ethyl acetate, the reaction liquid was transferred to a separating funnel and washed with water and brine. The resultant was dried over anhydrous sodium sulfate and then concentrated under reduced pressure. The resultant crude product was purified by silica gel column chromatography. Elution with a mixed solvent of toluene and ethyl acetate (67:33) gave a compound (9) (228.2 mg, 98% yield).

[α]_{D}−96.9° (c 1.0, chloroform)

¹H-NMR (CDCl₃): δ 8.11-8.09 (2H, m, Ar), 7.89-7.87 (2H, m, Ar), 7.81-7.78 (4H, m, Ar), 7.60-7.51 (4H, m, Ar), 7.44-7.14 (13H, m, Ar), 6.09 (1H, dd, J_{3,4}=10.3 Hz, J_{4,5}=10.0 Hz, H-4), 5.99 (1H, br d, J_{2,3}=3.4 Hz, H-2), 5.68 (1H, br s, CH₂CONH), 5.57 (1H, dd, J_{2,3}=3.4 Hz, J_{3,4}=10.3 Hz, H-3), 5.35 (1H, m, H-6^{Chol}), 5.25 (1H, br s, OCONH), 5.06 (1H, br s, H-1), 4.46 (1H, m, H-3^{Chol}), 3.93 (1H, dd, J_{5,6}=4.3 Hz, J_{gem}=11.7 Hz, H-6), 3.89-3.85 (2H, m, H-5, H-6), 3.39 (2H, br q, J=6.1 Hz, NHCH₂CH₂CO), 3.31 (2H, m, NHCH₂CH₂CH₂S), 2.79 (2H, m, NHCH₂CH₂CH₂S), 2.35-2.26 (4H, m, NHCH₂CH₂CO, H-4^{Chol}), 2.01-1.81 (7H, m, NHCH₂CH₂CH₂S, H-1eq^{Chol}, H-2eq^{Chol}, H-7eq^{Chol}, H-12eq^{Chol}, H-16eq^{Chol}), 1.58-0.90 (36H, m, ^{t}Bu, CH^{Chol}, CH₂^{Chol}, CH₃^{Chol}), 0.87 (3H, d, J=6.7 Hz, CH₃CH₂CH₃), 0.86 (3H, d, J=6.6 Hz, CH₃CH₂CH₃), 0.67 (3H, s, H-18^{Chol}).

ESI-TOF (high resolution): calcd for C₇₇H₉₈N₂NaO₁₁SSi [M+Na]⁺: 1309.6553. found; 1309.6556.

### Step [7]: Synthesis of N-(N-cholesteryloxycarbonyl-3-aminopropionyl)-3-aminopropyl 2,3,4-tri-O-benzoyl-1-thio-β-D-mannopyranoside (10)

To a solution of the compound (9) (112.3 mg) in tetrahydrofuran (1 mL) was slowly added acetic acid (0.05 mL) under an argon atmosphere at 0° C. To the mixed liquid was slowly added a solution (0.35 mL) of 1 M tetra-n-butylammonium fluoride in tetrahydrofuran at 0° C., and the mixture was stirred at room temperature for 2 days. After dilution with ethyl acetate, the reaction liquid was transferred to a separating funnel and washed with a saturated sodium bicarbonate aqueous solution, water, and brine. The resultant was dried over anhydrous sodium sulfate and then concentrated under reduced pressure. The resultant crude product was purified by silica gel column chromatography. Elution with a mixed solvent of toluene and ethyl acetate (25:75), subsequent elution with a mixed solvent of toluene and ethyl acetate (20:80), and subsequent elution with a mixed solvent of toluene and ethyl acetate (17:83) gave a compound (10) (87.3 mg, 95% yield).

[α]_{D}−135.6° (c 1.0, chloroform)

¹H-NMR (CDCl₃): δ 8.08-8.06 (2H, m, Ar), 7.94-7.92 (2H, m, Ar), 7.77-7.75 (2H, m, Ar), 7.61 (1H, dd, J=7.5, 7.4 Hz, Ar), 7.53-7.22 (8H, m, Ar), 6.55 (1H, br s, CH₂CONH), 5.99 (1H, m, H-2), 5.70 (1H, dd, J_{3,4}=10.1 Hz, J_{4,5}=8.8 Hz, H-4), 5.66 (1H, dd, J_{2,3}=3.2 Hz, J_{3,4}=10.1 Hz, H-3), 5.42-5.27 (2H, m, H-6^{Chol}, OCONH), 5.06 (1H, br s, H-1), 4.46 (1H, m, H-3^{Chol}), 3.92-3.84 (3H, m, H-5, H-6), 3.76 (1H, br s, OH), 3.48-3.36 (4H, m, NHCH₂CH₂CH₂S, NHCH₂CH₂CO), 2.86 (1H, m, NHCH₂CH₂CH₂S), 2.76 (1H, m, NHCH₂CH₂CH₂S), 2.43 (2H, br t, J=6.1 Hz, NHCH₂CH₂CO), 2.35-2.24 (2H, m, H-4^{Chol}), 2.01-0.90 (34H, m, NHCH₂CH₂CH₂S, CH^{Chol}, CH₂^{Chol}, CH3^{Chol}), 0.87 (3H, d, J=6.7 Hz, CH₃CH₂CH₃), 0.86 (3H, d, J=6.6 Hz, CH₃CH₂CH₃), 0.67 (3H, s, H-18^{Chol}).

ESI-TOF (high resolution): calcd for C₆₁H₈₀N₂NaO₁₁S [M+Na]⁺: 1071.5375. found; 1071.5375.

### Step [8]: Synthesis of N-(N-cholesteryloxycarbonyl-3-aminopropionyl)-3-aminopropyl 6-O-phospho-1-thio-β-D-mannopyranoside disodium salt (1)

To a solution of phosphorus oxychloride (0.09 mL) in pyridine (8 mL) was added dropwise a solution of the compound (10) (251.2 mg) in pyridine (4 mL) under an argon atmosphere at 0° C. over 2 hours (flow rate: 75 μL/min) through the use of a syringe pump. After the dropwise addition, the mixture was stirred at 0° C. for 15 minutes. After that, water (2.5 mL) was added thereto, and the mixture was stirred at 0° C. for 15 minutes. The reaction liquid was concentrated under reduced pressure and dried. After that, the residue was dissolved in tetrahydrofuran (5 mL) and methanol (7 mL). To the solution was added a solution of 1 M sodium methylate in methanol (4.78 mL) under an argon atmosphere at room temperature. The mixture was stirred at room temperature for 1 day, followed by dilution with water and dialysis. The aqueous solution was freeze-dried to give a compound (I) (201.4 mg, 98% yield).

[α]_{D}+19.1° (c 0.2, acetic acid)

¹H-NMR (CD₃CO₂D): δ 5.40 (1H, br s, H-6^{Chol}), 4.79 (1H, br s, H-1), 4.46 (1H, m, H-3^{Chol}), 4.27 (1H, dd, J_{5,6}=6.5 Hz, J_{gem}=9.8 Hz, H-6), 4.15 (1H, m, H-6), 4.08 (1H, br d, J_{2,3}=3.3 Hz, H-2), 3.84 (1H, dd, J_{3,4}=9.6 Hz, J_{4,5}=9.7 Hz, H-4), 3.76 (1H, dd, J_{2,3}=3.3 Hz, J_{3,4}=9.6 Hz, H-3), 3.57 (1H, m, H-5), 3.45-3.33 (4H, m, NHCH₂CH₂CH₂S, NHCH₂CH₂CO), 2.75 (2H, m, NHCH₂CH₂CH₂S), 2.52 (2H, m, NHCH₂CH₂CO), 2.40-2.28 (2H, m, H-4^{Chol}), 2.18-1.84 (7H, m, NHCH₂CH₂CH₂S, H-1eq^{Chol}, H-2eq^{Chol}, H-7eq^{Chol}, H-12eq^{Chol}, H-16eq^{Chol}), 1.67-0.95 (27H, m, CH^{Chol}, CH₂^{Chol}, CH₃^{Chol}), 0.88 (3H, d, J=6.6 Hz, CH₃CH₂CH₃), 0.88 (3H, d, J=6.6 Hz, CH₃CH₂CH₃), 0.72 (3H, s, H-18^{Chol}).

ESI-TOF (high resolution): calcd for C₄₀H₆₈N₂O₁₁PS [M]⁻: 815.4287. found; 815.4286.

### 2. Evaluation of Mannose-6-Phosphate-Modified Cholesterol Derivative-Containing Formulations for Physical Properties (FIG. 2 and FIG. 3)

In the production of mannose-6-phosphate-modified cholesterol derivative-containing liposomes and emulsions, first, various constituent lipids were dissolved in chloroform at various proportions (FIG. 2 and FIG. 3), and the solutions were each dispensed in a recovery flask. After that, the solvent was distilled off under reduced pressure with a rotary evaporator to prepare lipid films, which were dried under reduced pressure for 3 hours or more. To the dried films was added an optimal aqueous solution such as physiological saline, and the mixture was stirred with a shaking machine, sonicated for 10 minutes with a bath-type sonicator, and then sonicated for 3 minutes with a tip-type sonicator under nitrogen replacement. The resultant was subjected to filter sterilization with a polycarbonate membrane having a pore diameter of 0.45 μm. The concentrations of the liposomes and the emulsions were measured on the basis of the amount of a phospholipid or cholesterol.

After that, the produced liposomes and emulsions were evaluated for physicochemical properties by measuring a particle diameter and a surface charge. As a result, in each of the liposomes and the emulsions, the particle diameter was about 100 nm for all the lipid compositions, whereas the surface charge decreased depending on the content of the mannose-6-phosphate-modified cholesterol derivative.

It should be noted that in FIG. 2 and FIG. 3, M6P-Chol represents the mannose-6-phosphate-modified cholesterol derivative of the present invention manufactured according to FIG. 1.

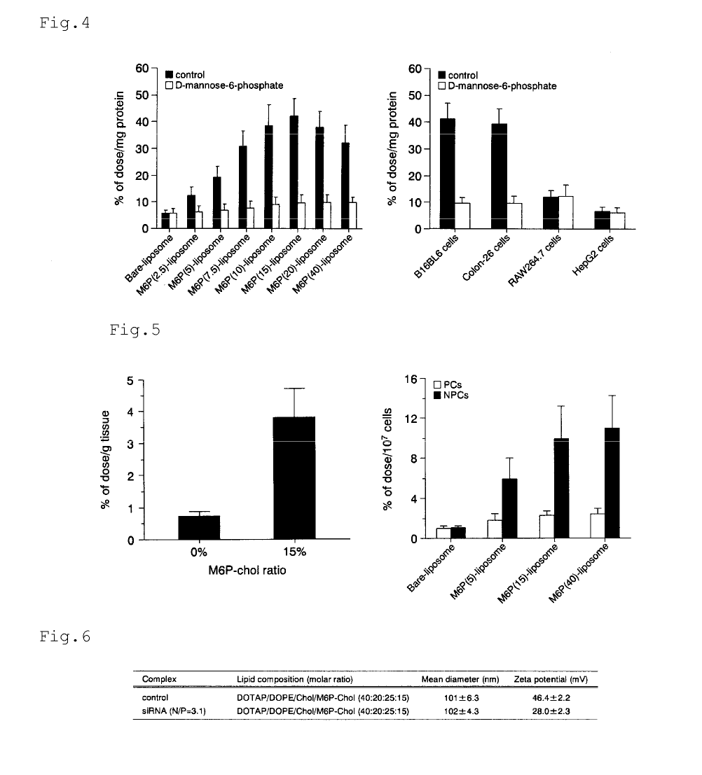

### 3. Evaluation of Mannose-6-Phosphate-Modified Cholesterol Derivative-Containing Liposomes for Intracellular Uptake Characteristics (FIG. 4)

Each of produced mannose-6-phosphate-modified cholesterol derivative-containing liposomes was confirmed to have a characteristic of recognition on a mannose-6-phosphate receptor serving as a target molecule, and investigated for its intracellular uptake mechanism. First, mannose-6-phosphate-modified cholesterol derivative-containing liposomes were produced by using a radiolabeled form ³H-labeled DSPC, and the mannose-6-phosphate receptor-mediated intracellular uptake of the mannose-6-phosphate-modified cholesterol derivative-containing liposomes was evaluated by using a melanoma-derived cancer cell line B16BL6 expressing the mannose-6-phosphate receptor. As a result of culture for 2 hours after the addition of the ³H-labeled liposomes, the intracellular uptake amount of the liposomes increased in a mannose-6-phosphate-modified cholesterol content-dependent manner and reached the maximum at a mannose-6-phosphate-modified cholesterol derivative content of 15% (FIG. 4: left). In addition, the addition of an excess amount of mannose-6-phosphate inhibited the intracellular uptake of the mannose-6-phosphate-modified cholesterol derivative-containing liposomes. This revealed that the intracellular uptake of the liposome formulation of the present invention was mediated by the mannose-6-phosphate receptor on the cell membrane (FIG. 4: left).

Further, the same evaluation was performed also in cells exhibiting different mannose-6-phosphate receptor-expressing characteristics. As a result, the mannose-6-phosphate receptor-mediated intracellular uptake was found in B16BL6 and colon-26 cells highly expressing the mannose-6-phosphate receptor. On the other hand, the mannose-6-phosphate receptor-mediated intracellular uptake was not found in RAW264.7 and HepG2 cells as mannose-6-phosphate receptor-non-expressing cells (FIG. 4: right).

### 4. Hepatic and Tumor Distribution Characteristics of Mannose-6-Phosphate-Modified Cholesterol Derivative-Containing Liposomes (FIG. 5)

Evaluations were made of B16BL6 cell-derived solid tumor and hepatic distribution characteristics of mannose-6-phosphate-modified cholesterol derivative-containing liposomes after the intravenous administration. As in the above-mentioned in-vitro experiment, mannose-6-phosphate-modified cholesterol derivative-containing liposomes were produced by using a radiolabeled form ³H-labeled DSPC, and intravenously administered to cancer-bearing mice, which were produced by transplanting B16BL6 cells exhibiting a high expression amount of the mannose-6-phosphate receptor under the back skin of C57BL/6 mice, at the time point when the tumor volume reached about 300 mm³. 24 hours after the intravenous administration of the formulations, the tumor tissues were excised, completely lysed with addition of a solubilizer, and then decolorized with addition of isopropanol and a 30% hydrogen peroxide solution. Further, hydrochloric acid was added for neutralization, and a scintillator was added to measure the radioactivity of ³H with a liquid scintillation counter. The resultant radioactivity was evaluated after normalization with an organ weight (g). As a result, 24 hours after the intravenous administration of the liposome formulations to the B16BL6-derived cancer-bearing mice, high tumor tissue distribution was found in the mannose-6-phosphate-modified cholesterol derivative-containing liposome administration group (FIG. 5: left).

In addition, it is known that the expression of the mannose-6-phosphate receptor is induced in hepatic stellate cells of hepatic cirrhosis mouse models. Thus, a carbon tetrachloride solution (2% in olive oil, 10 mL/kg) was intraperitoneally administered to C57BL/6 mice at frequent intervals of twice a week for 4 weeks to produce carbon tetrachloride-induced hepatic cirrhosis mouse models. The ³H-labeled liposome formulations were intravenously administered to the hepatic cirrhosis mouse models. 6 hours after the administration, the liver was fractionated into parenchymal cells (PCs) and non-parenchymal cells (NPCs) by collagenase perfusion, and the radioactivity of each fraction was evaluated after normalization with the number of cells. As a result, the distribution of the liposomes selective for the hepatic non-parenchymal cells (NPCs) as a hepatic stellate cell-containing fraction was found in the mannose-6-phosphate-modified cholesterol derivative-containing liposome administration group (FIG. 5: right).

[Application to siRNA Delivery]

### 5. Evaluation of Mannose-6-Phosphate-Modified Cholesterol Derivative-Containing Formulation for Physical Properties (FIG. 6)

In order to produce a mannose-6-phosphate-modified cholesterol derivative-containing liposome having cationic property capable of forming a complex with siRNA, various constituent lipids were dissolved in chloroform at the following constituent proportions (FIG. 6) and dispensed in a recovery flask, and then the solvent was distilled off under reduced pressure with a rotary evaporator to prepare a lipid film, which was dried under reduced pressure for 3 hours or more. A 5% glucose solution was added thereto, and the mixture was stirred with a shaking machine, sonicated for 10 minutes with a bath-type sonicator, and then sonicated for 3 minutes with a tip-type sonicator under nitrogen replacement. The resultant was subjected to filter sterilization with a polycarbonate membrane having a pore diameter of 0.45 μm. The concentration of the liposome was measured on the basis of the amount of a phospholipid or cholesterol. After that, in order to form a liposome/siRNA complex, siRNA against firefly luciferase and the mannose-6-phosphate-modified cholesterol derivative-containing cationic liposome were mixed in 5% dextrose at a charge ratio of 1.0:3.1 (−:+) to produce the complex.

In this case, firefly luciferase siRNA having the following sequences was used (A, G, C, U, and T represent adenosine, guanosine, cytidine, uridine, and thymidine, respectively, and X and dX represent a ribonucleotide and a deoxyribonucleotide, respectively (X represents any one of the abbreviations)).

Firefly luciferase siRNA:

Sense strand: CUUACGCUGAGUACUUCGAdTdT

Antisense strand: UCGAAGUACUCAGCGUAAGdTdT

The produced formulations were evaluated for physical properties by measuring a particle diameter and a surface charge. As a result, the particle diameter was about 100 nm irrespective of the siRNA complexation, whereas the surface charge was reduced by the siRNA complexation.

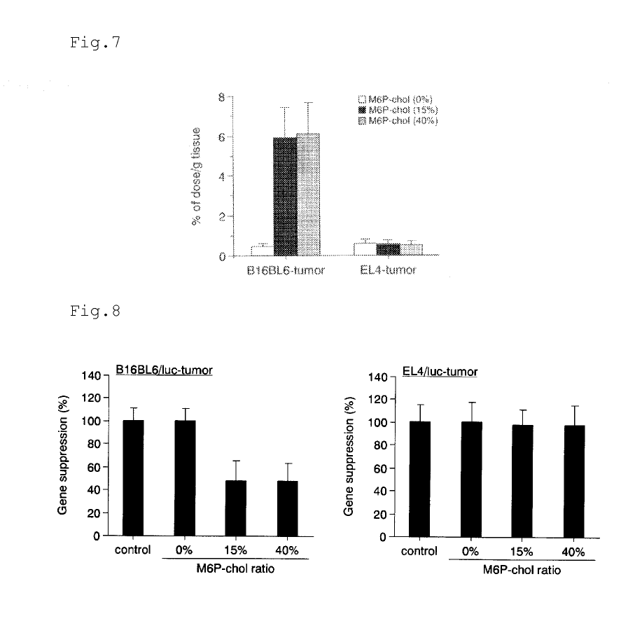

6. Tumor Distribution Characteristics of siRNA (FIG. 7) and Suppressing Effects on Gene Expression (FIG. 8) by Mannose-6-Phosphate-Modified Cholesterol Derivative-Containing Liposome/siRNA Complexes

Evaluations were made of the tumor tissue distribution of siRNA by the intravenous administration of mannose-6-phosphate-modified cholesterol derivative-containing liposome/siRNA complexes. First, mannose-6-phosphate-modified cholesterol derivative-containing liposome/siRNA complexes were produced by using siRNA against firefly luciferase (firefly luciferase siRNA) labeled with a fluorescent dye Alexa-488, and the formulations were intravenously administered (50 μg in terms of siRNA) to cancer-bearing mice, which were produced by transplanting B16BL6 cells and EL4 cells under the back skin of C57BL/6 mice, at the time point when the tumor volume reached about 300 mm³. 24 hours after the administration, the tumor tissues were excised and lysed with addition of a tissue lysis solution and with a homogenizer. After that, the resultant tissue lysates were subjected to a freeze-thawing operation with liquid nitrogen and in a hot water bath at 37° C., followed by centrifugation. The intensities of fluorescence in the resultant supernatants were measured and evaluated after normalization with an organ weight (g). As a result, 24 hours after the intravenous administration, high tumor tissue distribution of siRNA by the administration of the mannose-6-phosphate-modified cholesterol derivative-containing liposome/siRNA complexes was found in the B16BL6-derived solid tumor as mannose-6-phosphate receptor-expressing cells. On the other hand, no increase in tumor tissue distribution of siRNA was found in the EL4-derived solid tumor as mannose-6-phosphate receptor-non-expressing cells. Thus, an increase in distribution of siRNA into the mannose-6-phosphate receptor-expressing cancer cells was able to be achieved (FIG. 7).

Next, evaluations were made of suppressing effects on gene expression in tumor tissues by the intravenous administration of the mannose-6-phosphate-modified cholesterol derivative-containing liposome/siRNA complexes. The mannose-6-phosphate-modified cholesterol derivative-containing liposome/siRNA complexes (50 μg in terms of siRNA) were intravenously administered to cancer-bearing mice, which were produced by transplanting B16BL6/Luc cells and EL4/Luc cells as cell lines stably expressing firefly luciferase under the back skin of C57BL/6 mice, at the time point when the tumor volume reached about 300 mm³, to thereby deliver siRNA against firefly luciferase. As a result, 24 hours after the intravenous administration of the formulations, high suppressing effects on gene expression were found in the B16BL6/luc-derived solid tumor as mannose-6-phosphate receptor-expressing cancer cells. On the other hand, no suppressing effect on gene expression was found in the EL4/luc-derived solid tumor as mannose-6-phosphate receptor-non-expressing cells. This indicated that remarkable suppressing effects on gene expression were obtained by virtue of high distribution of siRNA into the mannose-6-phosphate receptor-expressing cancer cells (FIG. 8).

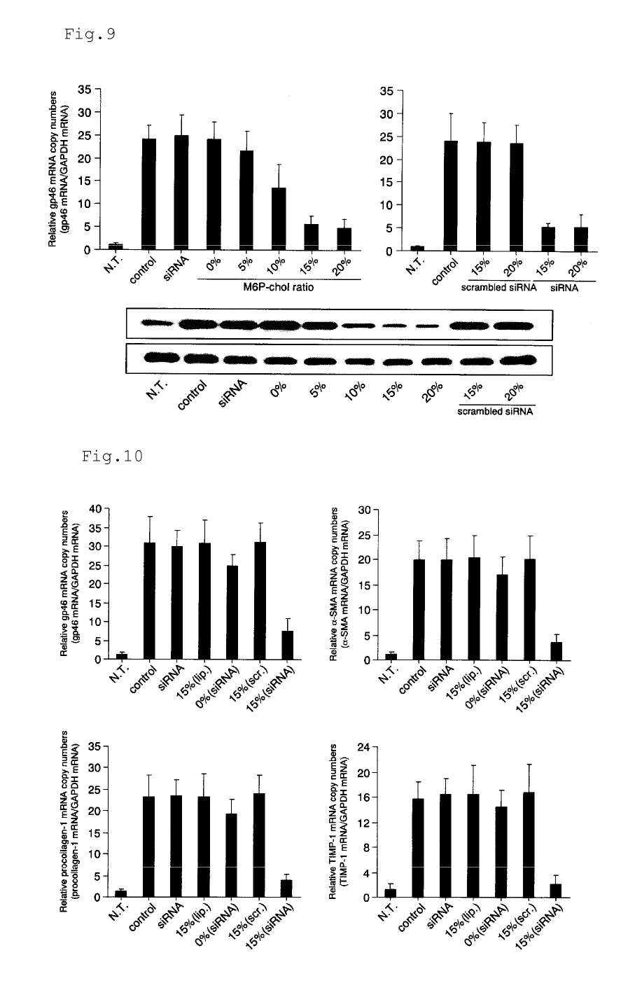

7. Suppressing Effects on Gp46 Expression in Carbon Tetrachloride-Induced Hepatic Cirrhosis Mouse Models (FIG. 9) and Therapeutic Effects on Hepatic Cirrhosis (FIG. 10) by mannose-6-phosphate-modified cholesterol derivative-containing Liposome/Gp46 siRNA Complexes

It is known that the expression of the mannose-6-phosphate receptor is induced in hepatic stellate cells of hepatic cirrhosis mouse models. Thus, a carbon tetrachloride solution (2% in olive oil, 10 mL/kg) was intraperitoneally administered to C57BL/6 mice at frequent intervals of twice a week for 4 weeks to produce carbon tetrachloride-induced hepatic cirrhosis mouse models, and evaluations were made of suppressing effects on gp46 expression in liver in the carbon tetrachloride-induced hepatic cirrhosis mouse models by the intravenous administration of the mannose-6-phosphate-modified cholesterol derivative-containing liposome/gp46 siRNA complexes. In this case, gp46 is a chaperone protein (HSP47 in humans) involved in collagen production, and there is a report that its expression is induced under the condition of hepatic cirrhosis. Collagen production is suppressed by the suppression of the gene to achieve the suppression and treatment of hepatic cirrhosis progression. gp46 siRNA and the mannose-6-phosphate-modified cholesterol derivative-containing cationic liposomes were mixed at a charge ratio of 1.0:3.1 (−:+) to produce mannose-6-phosphate-modified cholesterol derivative-containing liposome/gp46 siRNA complexes, and the gp46 siRNA complexes (50 μg in terms of gp46 siRNA) were intravenously administered.

In this case, gp46 siRNA and scrambled siRNA having the following sequences were used (A, G, C, U, and T represent adenosine, guanosine, cytidine, uridine, and thymidine, respectively, and X and dX represent a ribonucleotide and a deoxyribonucleotide, respectively (X represents any one of the abbreviations)).

gp46 siRNA:

Sense strand: GUUCCACCAUAAGAUGGUAGACAACAGdTdT
Antisense strand: GUUGUCUACCAUCUUAUGGUGGAACAUdTdT

Scrambled siRNA:

Sense strand: CGAUUCGCUAGACCGGCUUCAUUGCAGdTdT
Antisense strand: GCAAUGAAGCCGGUCUAGCGAAUCGAUdTdT

As a result of the experiment, 24 hours after the administration, gp46 whose expression was induced in carbon tetrachloride-induced hepatic cirrhosis was suppressed at mRNA and protein levels by the intravenous administration of the mannose-6-phosphate-modified cholesterol derivative-containing liposome/gp46 siRNA (FIG. 9). In addition, the suppressing effect on gp46 expression increased depending on a mannose-6-phosphate-modified cholesterol derivative content and reached the maximum at a mannose-6-phosphate-modified cholesterol derivative content of from 15 to 20% (FIG. 9).

Further, evaluations were made of the influences of the suppressed expression of gp46 by the mannose-6-phosphate-modified cholesterol derivative-containing liposome/gp46 siRNA on various markers in carbon tetrachloride-induced hepatic cirrhosis. In this case, α-smooth muscle actin (α-SMA) is a marker molecule of activated hepatic stellate cells involved in collagen production under the condition of hepatic cirrhosis, and procollagen-1 is a precursor for collagen leading to hepatic fibrosis and hepatic cirrhosis. In addition, a tissue inhibitor of metalloproteinase-1 (TIMP-1) is a tissue inhibitor of metalloprotease whose expression is induced under the condition of hepatic cirrhosis and which is involved in collagen decomposition and the like. The mannose-6-phosphate-modified cholesterol derivative-containing liposome/gp46 siRNA complexes were produced at a charge ratio of 1.0:3.1 (−:+) and intravenously administered at a dose of 50 μg in terms of gp46 siRNA at frequent intervals (twice a week for 3 weeks, carbon tetrachloride continued to be intraperitoneally administered twice a week for this period), and the expression amounts of gp46, α-SMA, procollagen-1, and TIMP-1 in liver were evaluated. The results revealed that the suppressed expression of gp46 by the intravenous administration of the mannose-6-phosphate-modified cholesterol derivative-containing liposome/gp46 siRNA remarkably suppressed any of the factors (FIG. 10).

[Application to Anticancer Agent Delivery]

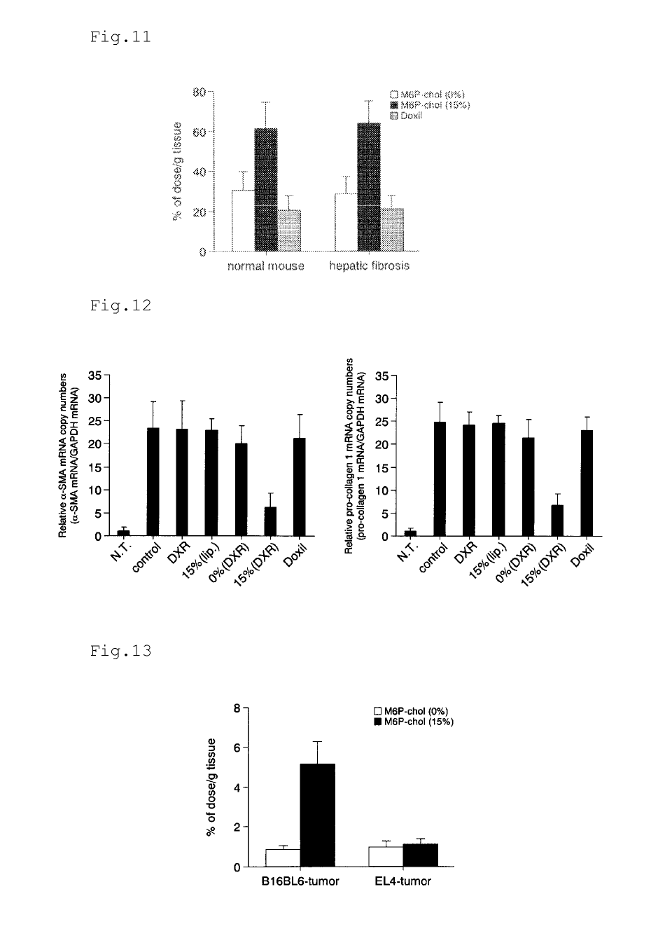

### 8. Hepatic Distribution Characteristics of Doxorubicin (FIG. 11) and Therapeutic Effect on Hepatic Cirrhosis (FIG. 12) by Doxorubicin-Encapsulated Mannose-6-Phosphate-Modified Cholesterol Derivative-Containing Liposome

The hepatic distribution of doxorubicin by the intravenous administration of a doxorubicin-encapsulated mannose-6-phosphate-modified cholesterol derivative-containing liposome was evaluated by using normal mice and hepatic cirrhosis mouse models. In order to produce a mannose-6-phosphate-modified cholesterol derivative-containing liposome capable of being complexed with doxorubicin, various lipids were dissolved in chloroform and dispensed in a recovery flask, and then the solvent was distilled off under reduced pressure with a rotary evaporator to prepare a lipid film, which was dried under reduced pressure for 3 hours or more. A 250 mM ammonium sulfate aqueous solution was added thereto. After stirring with a shaking machine, the mixture was sonicated for 10 minutes with a bath-type sonicator and then sonicated for 3 minutes with a tip-type sonicator under nitrogen replacement. The resultant was subjected to filter sterilization with a polycarbonate membrane having a pore diameter of 0.45 μm. The complexation with doxorubicin was performed by a remote-loading method. Specifically, the produced liposome solution was subjected to gel filtration with a column filled with a Sephadex G-25 using PBS (pH 8.0) as a developing solvent. The liposome solution in which the external aqueous phase was replaced with PBS (pH 8.0) and doxorubicin were mixed at a ratio of liposome:doxorubicin=10:1 (mol/mol) and shaken at 60° C. for 1 hour to encapsulate doxorubicin into the liposome. In this experiment, Doxil, a doxorubicin-encapsulated polyethylene glycol-modified liposome formulation clinically used as an anticancer agent, was used as a comparative control. In addition, carbon tetrachloride-induced hepatic cirrhosis mouse models produced by intraperitoneally administering a carbon tetrachloride solution (2% in olive oil, 10 mL/kg) to C57BL/6 mice at frequent intervals of twice a week for 4 weeks were used as the hepatic cirrhosis mouse models.

The doxorubicin-encapsulated mannose-6-phosphate-modified cholesterol derivative-containing liposome (4 mg/kg in terms of doxorubicin) was intravenously administered to normal mice and carbon tetrachloride-induced hepatic cirrhosis mouse models, and 6 hours after the administration, the tumor tissues were excised and lysed with addition of a tissue lysis solution and with a homogenizer. After that, the resultant tissue lysates were subjected to a freeze-thawing operation with liquid nitrogen and in a hot water bath at 37° C., followed by centrifugation. The intensities of fluorescence derived from doxorubicin in the resultant supernatants were measured and evaluated after normalization with an organ weight (g).

As a result, in each of the normal mice and hepatic cirrhosis mouse models, remarkably high hepatic distribution of doxorubicin was found to be achieved by delivering doxorubicin using the mannose-6-phosphate-modified cholesterol derivative-containing liposome. The amount of hepatic distribution of doxorubicin is extremely high even in comparison to the doxorubicin-encapsulated polyethylene glycol-modified liposome formulation (Doxil), which is in practical use in the clinical setting at present. Thus, an increase in hepatic distribution of doxorubicin was able to be achieved by the mannose-6-phosphate-modified cholesterol derivative-containing liposome (FIG. 11).

Further, evaluations were made of the influences of the intravenous administration of the doxorubicin-encapsulated mannose-6-phosphate-modified cholesterol derivative-containing liposome on α-smooth muscle actin (α-SMA) and procollagen-1, which were enhanced in carbon tetrachloride-induced hepatic cirrhosis. The doxorubicin-encapsulated mannose-6-phosphate-modified cholesterol derivative-containing liposome was intravenously administered to carbon tetrachloride-induced hepatic cirrhosis mouse models at a dose of 4 mg/kg in terms of doxorubicin at frequent intervals (twice a week for 3 weeks, carbon tetrachloride continued to be intraperitoneally administered twice a week for this period), and the expression amounts of α-SMA and procollagen-1 in liver were evaluated. The results revealed that the expression levels of both the factors were suppressed by the doxorubicin-encapsulated mannose-6-phosphate-modified cholesterol derivative-containing liposome (FIG. 12). The results indicated that doxorubicin was introduced into hepatic stellate cells by the mannose-6-phosphate-modified cholesterol derivative-containing liposome, revealing that the formulation was applicable to hepatic cirrhosis treatment.

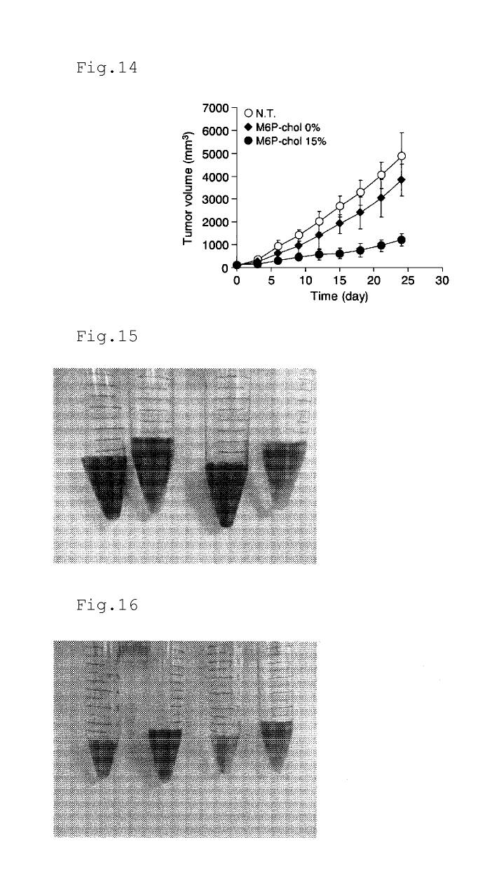

### 7. Tumor Tissue Distribution Characteristics of Doxorubicin (FIG. 13) and Antitumor Effect (FIG. 14) by Doxorubicin-Encapsulated Mannose-6-Phosphate-Modified Cholesterol Derivative-Containing Liposome

The tumor tissue distribution of doxorubicin by the intravenous administration of a doxorubicin-encapsulated mannose-6-phosphate-modified cholesterol derivative-containing liposome was evaluated by using B16BL6 and EL4-derived solid tumor mouse models. A production method for the doxorubicin-encapsulated mannose-6-phosphate-modified cholesterol derivative-containing liposome is as described above. In addition, the solid tumor mouse models were produced by transplanting B16BL6 cells and EL4 cells under the back skin of C57BL/6 mice.

The doxorubicin-encapsulated mannose-6-phosphate-modified cholesterol derivative-containing liposome (4 mg/kg in terms of doxorubicin) was intravenously administered to cancer-bearing mice in which the tumor volume reached about 300 mm³. As a result, in the B16BL6-derived solid tumor as mannose-6-phosphate receptor-expressing cells, high tumor tissue distribution of doxorubicin was found to be achieved by the administration of the doxorubicin-encapsulated mannose-6-phosphate-modified cholesterol derivative-containing liposome. On the other hand, in the EL4-derived solid tumor as mannose-6-phosphate receptor-non-expressing cells, no increase in tumor tissue distribution of doxorubicin was found. Thus, an increase in distribution of doxorubicin into the mannose-6-phosphate receptor-expressing cancer cells was able to be achieved (FIG. 13)

Further, the antitumor effect of the intravenous administration of the doxorubicin-encapsulated mannose-6-phosphate-modified cholesterol derivative-containing liposome was evaluated by using B16BL6-derived solid tumor mouse models. The doxorubicin-encapsulated mannose-6-phosphate-modified cholesterol derivative-containing liposome was intravenously administered to the solid tumor mouse models, which were produced by transplanting B16BL6 cells under the back skin of C57BL/6 mice, at a single dose of 4 mg/kg in terms of doxorubicin at the time point when the tumor volume reached about 100 mm³, and the tumor volume after the administration was measured chronologically. As a result, the administration of the doxorubicin-encapsulated mannose-6-phosphate-modified cholesterol derivative-containing liposome was found to exhibit a remarkable tumor growth-suppressing effect on the B16BL6 solid tumor, whereas such effect was not found in an unmodified liposome. The results revealed that the doxorubicin-encapsulated mannose-6-phosphate-modified cholesterol derivative-containing liposome exhibited an antitumor effect specific for mannose-6-phosphate receptor-expressing cancer cells (FIG. 14).

### Example 2

Indocyanine green and hematoporphyrin were each encapsulated into a mannose-6-phosphate (M6P)-modified liposome.

### (1) Preparation of Indocyanine Green-Encapsulated Mannose-6-Phosphate (M6P)-Modified Liposome

### Methods

### 1. Preparation of Indocyanine Green (ICG)-Encapsulated M6P-Modified Liposome

Lipids were mixed in chloroform according to the following composition, and then the solvent was removed with an evaporator. 1,2-Distearoyl-sn-glycero-3-phosphocholine (DSPC):cholesterol:M6P-cholesterol=60:40-x:x (molar ratio x=0 or 15, total lipid: 40 mg)

The resultant was left to stand still in a desiccator overnight. Then, 4 ml of an ICG aqueous solution (1 mg/ml in DI water) were added thereto, and the mixture was shaken in a water bath at 65° C. for 30 minutes. After that, the dispersion was sonicated for 10 minutes in a bath-type sonicator and for 3 minutes in a tip-type sonicator to give an ICG-encapsulated M6P-modified liposome. The resultant liposome solution was filtered with a 0.45-μm syringe filter and used in the following experiment.

### 2. Measurement of ICG Encapsulation Ratio of ICG-Encapsulated M6P-Modified Liposome

The ICG-encapsulated M6P-modified liposome was filtered with a PD-10 column to separate an external layer. It should be noted that distilled water was used as a solvent. After that, each of the liposome solution prepared in 1 and the liposome solution in which the external layer was separated this time was measured for its absorbance at a wavelength of 780 nm, and determined for its ICG concentration from a calibration curve. In addition, the two liposome solutions were determined for lipid concentrations with a phospholipid quantification kit, and from the two values, ICG concentrations and ICG encapsulation ratios per lipid were determined.

Results

The resultant liposomes were as shown in FIG. 15.

It is found that ICG in the external layer was removed by filtration with a PD-10 column, and as a result, the color of the solution became pale.

TABLE 1
Encapsulation Ratio

| 0 | 15 |
|---|---|
| 46.16% | 58.16% |

The encapsulation ratios were as shown in Table 1. The numeral 0 represents an unmodified liposome and the numeral 15 represents an M6P liposome containing M6P-cholesterol at 15 mol %.

It was confirmed that ICG was able to be encapsulated into the M6P liposome.

### (2) Preparation of Hematoporphyrin-Encapsulated Mannose-6-Phosphate (M6P)-Modified Liposome

### Methods

### 1. Preparation of Hematoporphyrin (Hp)-Encapsulated M6P-Modified Liposome

Lipids were mixed in chloroform according to the following composition, 2 ml of an Hp solution (1 mg/ml in methanol) were added thereto, and then the solvent was removed with an evaporator. 1,2-Distearoyl-sn-glycero-3-phosphocholine (DSPC):cholesterol:M6P-cholesterol=60:40-x:x (molar ratio x=0 or 15, total lipid: 20 mg)

The resultant was left to stand still in a desiccator overnight. Then, 4 ml of distilled water were added thereto, and the mixture was shaken in a water bath at 65° C. for 30 minutes. After that, the dispersion was sonicated for 10 minutes in a bath-type sonicator and for 3 minutes in a tip-type sonicator to give an Hp-encapsulated M6P-modified liposome. The resultant liposome solution was filtered with a 0.45-μm syringe filter and used in the following experiment.

### 2. Measurement of Hp Encapsulation Ratio of Hp-Encapsulated M6P-Modified Liposome

The measurement was performed in the same manner as in the ICG-encapsulated liposome. The Hp-encapsulated M6P-modified liposome was filtered with a PD-10 column to separate an external layer. It should be noted that distilled water was used as a solvent. After that, each of the liposome solution prepared in 1 and the liposome solution in which the external layer was separated this time was measured for its absorbance at a wavelength of 405 nm, and determined for its Hp concentration from a calibration curve. In addition, the two liposome solutions were determined for lipid concentrations with a phospholipid quantification kit, and from the two values, Hp concentrations and Hp encapsulation ratios per lipid were determined.

### Results

The resultant liposomes were as shown in FIG. 16.

It is found that Hp remaining in the external layer was removed by filtration with a PD-10 column as in ICG, and as a result, the color of the solution became pale.

TABLE 2
Encapsulation Ratio

| 0 | 15 |
|---|---|
| 74.43% | 83.03% |

The encapsulation ratios were as shown in Table 2. The numeral 0 represents an unmodified liposome and the numeral 15 represents an M6P liposome containing M6P-cholesterol at 15 mol %. It was found that Hp was encapsulated into the M6P liposome.

### CONCLUSION

It was revealed that indocyanine green or hematoporphyrin was able to be encapsulated into the M6P-modified liposome. In the future, applications to the fluorescence imaging of cancer cells highly expressing the M6P receptor and a sonodynamic therapy can be expected.

### INDUSTRIAL APPLICABILITY

The formulation of the present invention is useful as a therapeutic drug for hepatic cirrhosis, an anticancer agent, a cell-selective reagent for introducing a drug or a nucleic acid (reagent for research), or the like.

1. A compound, which is represented by the following general formula (1): 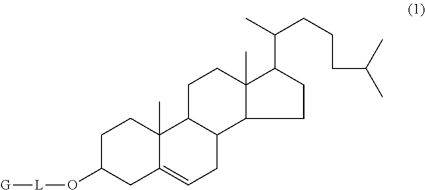
where:
G represents a mannose-6-phosphate residue;
L represents a divalent linker group represented by —X—R—Y—;
X represents O or S;
Y represents an alkylene group having from 1 to 6 carbon atoms, a cycloalkylene group having from 3 to 6 carbon atoms, an arylene group, an aralkylene group, —NHCO—, —O—CO—, or —CO—; and
R represents:
when Y represents an alkylene group having from 1 to 6 carbon atoms, a cycloalkylene group having from 3 to 6 carbon atoms, an arylene group, or an aralkylene group, a single bond or R1-R2 where R1 represents an alkylene group having from 1 to 6 carbon atoms, a cycloalkylene group having from 3 to 6 carbon atoms, an arylene group, or an aralkylene group and R2 represents —NHCO—, —CONH—, —O—, —S—, —NHCOO—, —OCONH—, —CO—, —COO—, or —O—CO—, or a polyether group; and
when Y represents —NHCO—, —O—CO—, or —CO—, R1 or R1-R2-R1 where R1's are identical to or different from each other and each represents an alkylene group having from 1 to 6 carbon atoms, a cycloalkylene group having from 3 to 6 carbon atoms, an arylene group, or an aralkylene group and R2 represents —NHCO—, —CONH—, —O—, —S—, —NHCOO—, —OCONH—, —CO—, —COO—, or —O—CO—.

2. (canceled)

3. The compound according to claim 1, wherein the linker group is represented by the following general formula:
—X—(CH₂)m-NHCO(CH₂)n-NHCO—
where:
X represents S or O;
m represents an integer of from 2 to 6; and
n represents an integer of from 2 to 6.

4. A mannose-6-phosphate-modified cholesterol derivative-containing formulation, comprising:
a liposome comprising the compound according to claim 1; and
a physiologically active substance complexed with the liposome.

5. The formulation according to claim 4, wherein the physiologically active substance comprises a therapeutic drug for hepatic cirrhosis, hepatitis, hepatic fibrosis, cancer, diabetes, or a lysosomal disease.

6. The formulation according to claim 4, wherein the physiologically active substance is a drug, a protein, or a nucleic acid.

7. The formulation according to claim 4, wherein the physiologically active substance is an anticancer agent, plasmid DNA/RNA, antisense DNA, an aptamer, siRNA, shRNA, or miRNA.

8. The formulation according to claim 4, wherein the physiologically active substance comprises an organic fluorescent dye.

9. The formulation according to claim 6, wherein the physiologically active substance is an anticancer agent, plasmid DNA/RNA, antisense DNA, an aptamer, siRNA, shRNA, or miRNA.

10. The formulation according to claim 6, wherein the physiologically active substance comprises an organic fluorescent dye.

11. The formulation according to claim 5, wherein the physiologically active substance is a drug, a protein, or a nucleic acid.

12. The formulation according to claim 11, wherein the physiologically active substance is an anticancer agent, plasmid DNA/RNA, antisense DNA, an aptamer, siRNA, shRNA, or miRNA.

13. The formulation according to claim 11, wherein the physiologically active substance comprises an organic fluorescent dye.

14. A mannose-6-phosphate-modified cholesterol derivative-containing formulation, comprising:
a liposome comprising the compound according to claim 3; and
a physiologically active substance complexed with the liposome.

15. The formulation according to claim 14, wherein the physiologically active substance comprises a therapeutic drug for hepatic cirrhosis, hepatitis, hepatic fibrosis, cancer, diabetes, or a lysosomal disease.

16. The formulation according to claim 15, wherein the physiologically active substance is a drug, a protein, or a nucleic acid.

17. The formulation according to claim 16, wherein the physiologically active substance is an anticancer agent, plasmid DNA/RNA, antisense DNA, an aptamer, siRNA, shRNA, or miRNA.

18. The formulation according to claim 16, wherein the physiologically active substance comprises an organic fluorescent dye.

19. The formulation according to claim 14, wherein the physiologically active substance is a drug, a protein, or a nucleic acid.

20. The formulation according to claim 19, wherein the physiologically active substance is an anticancer agent, plasmid DNA/RNA, antisense DNA, an aptamer, siRNA, shRNA, or miRNA.

21. The formulation according to claim 20, wherein the physiologically active substance comprises an organic fluorescent dye.
